(19) United States
(12) Patent Application Publication
(10) Pub. No.: US 2012/0172749 A1
(43) Pub. Date: Jul. 5, 2012

(54) Laryngeal Mask Airway (LMA) with Integrated Core Temperature Monitor and Display

(76) Inventors: KongYuan HE, Encino, CA (US); Fanping Wang, Encino, CA (US)

(21) Appl. No.: 13/419,307

(22) Filed: Mar. 13, 2012

Related U.S. Application Data
(63) Continuation-in-part of application No. 12/635,128, filed on Dec. 10, 2009, now abandoned.

(51) Int. Cl. A61B 5/01; A61M 16/04

(52) U.S. Cl. 600/549

(57) ABSTRACT

This is a device for use in clinical, surgical and intro-operative patient core temperature monitoring, which utilizes an artificial airway to integrate a temperature probe, removing the need for an external cord for connecting to an external display unit, while the LMA is used for patients who are under general anesthesia. Consequently, accurate monitoring of core temperature can be achieved more easily. As a result, the medical complication and risk fact associated with other traditional ways of temperature monitoring is greatly reduced.

### RELATED APPLICATION

This application is a Continuation-In-Part (CIP) application, under 37 CFR 1.53(b), of a prior parent application #12/635,128, by the same inventors, to supplement the disclosure matter related to the prior application. Present CIP application claims priority of the filing date of Dec. 10, 2009, per the requirements of 35 U.S.C. §120 and 37 C.F.R. 1.78.

### FIELD AND BACKGROUND OF THE INVENTION

The present invention relates generally to clinical, surgical and intro-operative monitoring of patients' temperature and especially who undergo general anesthesia. Particularly, the present invention provides a means for accurate measurement of core body temperature of patients, and thus provides better monitoring of patients' physical condition.

Traditionally, clinical and surgical temperature measurement and monitoring is done by surface measurement or a specially designed intrusive means, usually some type of PA (Pulmonary Artery) catheter, esophageal, rectal or urinal catheterization, to gain access to patients' inner body chamber to get temperature reading. The most common one is to measure the esophageal temperature by placing probe of roughly 45 cm into that part of the esophagus to measure core temperature.

Normally, an anesthesiologist places an esophageal probe after the patient is intubated under general anesthesia. The esophageal probe is connected to a central monitor through a special cord, and causes the temperature reading to be displayed on a screen of the external monitor.

The disadvantage of this traditional method includes:

1. The procedure of placing/inserting the esophageal probe into a patient's body takes additional time and effort. Sometimes it can be difficult and does add potential risk factors to the patients.

2. The esophageal probe cannot be placed in patients who relied upon the use of a regular LMA (Laryngeal Mask Airway). In the typical practice were LMA is used, doctors need to apply a skin-sensing strip or other means of temperature monitoring to patients for checking on patients' temperature. Apparently, the skin temperature does not give accurate patient information related to the patients' core body temperature, in response to any medical treatment, surgery, administration of drugs, or affected by the ambient temperature changes. If other means are used, there is concern for the accuracy as well.

3. Placing esophageal probe may sometimes cause sore throat or even oral injury.

4. It takes up additional oral space, which could be inconvenient for certain surgeries.

5. An external cord going from the patient to an external monitor is needed, which causes inconvenience to medical persons, including anesthesiologist, and thus also increases risk factors for tripping people around the patient and even causing accidental extubation, and is a potential threat to patient, consequently.

6. The esophageal temperature probes used associated with this traditional method is a bio-waste.

All these disadvantages prompted the improvements proposed by present invention.

### SUMMARY OF PRESENT INVENTION

An advantage of present invention is that the measurement of core body temperature will be taken from upper airway, instead of the esophagus. The measuring device is the adapted LMA described herein and thus is a lot simpler and safer to use for surgical and intro-operative purposes.

By obtaining vital signs, such as core temperature from the patient's airway, the process is made simpler and helps to reduce costs of extra procedures for monitoring temperature.

The important feature is to integrate one or more temperature probes to the LMA, removing the need to insert the esophageal tube. Additionally, precise core temperature can be monitored even when the patients are under general anesthesia with LMA.

A small display unit is integrally built to the outside portion of the adapted LMA device, so that medical service personnel, including nurses, doctors, anesthesiologists, will be able to check on patients' core temperature and provide needed responses, even without the help of an externally-connected display module.

Moreover, as applied to non-disposable LMA devices, by having a 2-unit construction, where only the in-body portion needs to be sterilized and the outside portion will be connected at time when it's ready for use, the bio-waste is reduced to only the in-body portion. It will reduce the cost of both reusable and disposable LMA, as the display unit can be repeatedly used until it runs out of battery power.

The 2-unit construction also helps to add more functions to LMA device, instead of placing another invasive devices into a patient's body.

### DESCRIPTION OF THE DRAWINGS

The accompanying drawings, which are incorporated in and constitute a part of this specification, illustrate the preferred embodiments of the invention and together with the description, serve to explain the principles of the invention, but are not intended to limit the scope of present invention to the extent present invention is applicable.

FIG. 1 shows the overall structure of the LMA integrated temperature monitor and display device.

FIG. 2 shows an architectural view of first embodiment of present invention.

FIG. 3 shows a cut-out structural view of present invention where the in-body portion temperature-sensing monitor can be optionally connected to an external monitor device, as compared to the integrated display unit (the second portion, see details later.)

FIGS. 4a and 4b show the two-unit construction of present invention.

### DETAILED DESCRIPTION OF PREFERRED EMBODIMENT

The device of present invention is made up of two major portions, the first portion is a in-body portion, having a thermo-couple temperature probe with conductive signal wires embedded inside the tubing wall of the LMA. The second portion contains a temperature display unit with digital readout, a printed circuit board and a battery (inside a battery compartment). These two portions can stay separated until ready for use.

For sterilization of the reusable LMA, only the first portion needs to be sterilized, since only this in-body portion will be place inside a patient's body.

The inside end of the LMA device, where the temperature probe is located, will be sitting in the pharyngeal space, outside of trachea, and will obtain the patient's core temperature based upon the posterior pharyngeal wall sensing and reading.

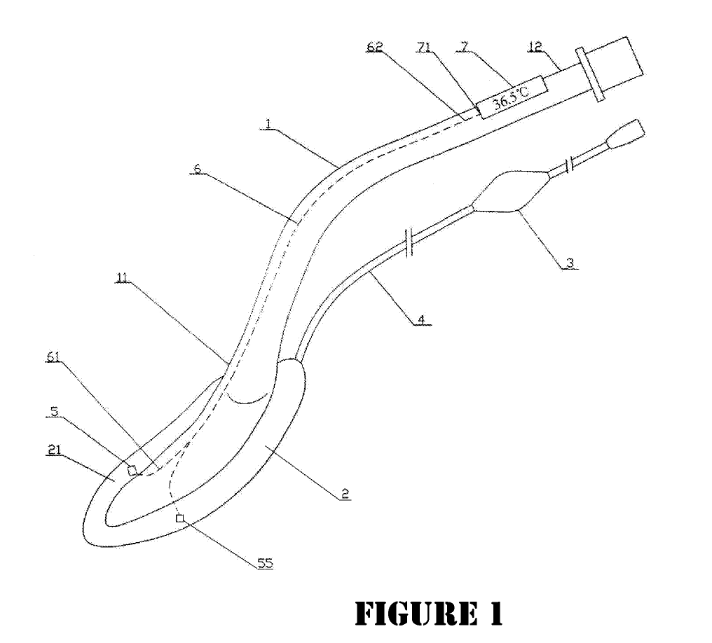

Referring to FIG. 1, where an air tube 1 has an inside end 11 and an outside end 12. FIG. 1 generally take the form of a regular LMA, which consists of an inflatable, elliptical mask (also known as an “air sac”, or “cuff”) at distal end, a tube (the air tube 1 referred herein) connecting to it.

The term “inside” and “outside” will be used in reference to the fact that the device of present invention's “inside” end will be placed inside a patient's oral cavity, that is, into the upper airway of a patient. The term “outside” will be used to describe the fact that some portion of the device in present invention will stay outside of a patient's body, during medical application/use.

An air sac 2 is located towards the inside end 11. In general LMA jargon, said air sac 2 is also referred to as a cuff.

A temperature display unit 7 is made up of a digital display unit, a battery compartment (for receiving a suitable battery) and a switch with associated printed circuit board. Said temperature display unit 7 is located near the outside end 12 of the air tube 1.

A temperature probe 5 is located on the sac wall 21 of said air sac 2.

An air pump 3 is attached, via a pumping tube 4, to the air sac 2, so that the air tube 1 looks like it has something “forking” out, with one fork going to the air pump 3 and the other fork going to the temperature display unit 7. Such air pump 3 may be also referred to as “pilot balloon” in the medical field.

Alternatively, two or more temperature probes 5 may be placed evenly spaced around the perimeter edge of the sac wall 21, so that the core temperature readout may be obtained by the averaged measurements, for more accurate core temperature monitoring.

The preferred locations of temperature probes 5 will be the perimeter edges along the dorsal side of said air sac 2, so that the probe(s) 5 will be in contact with the mucosa at the hypopharyngeal area for the LMA usage.

A preferred embodiment for the location of the temperature probe would be about one-third of the way in, from the distal tip of said air sac 2. The measurement of “one-third” would be taken from the general length-wise direction for the air sac 2.

A signal wire 6 is placed inside the air tube 1, or placed under a thin layer material connecting the temperature probe(s) 5 to the connecting socket 8, which then allows connection to either a display unit 7 or to an external monitor 9.

Note that the signal wire 6 should be a pair of “wires”, although it is used in its singular form herein. The implementation of a signal wire 6 is known art and need no disclosure by present application, and does not constitute any novelty part of present application, except to the extent that it forms part of the complete disclosure in combination with other parts of present invention.

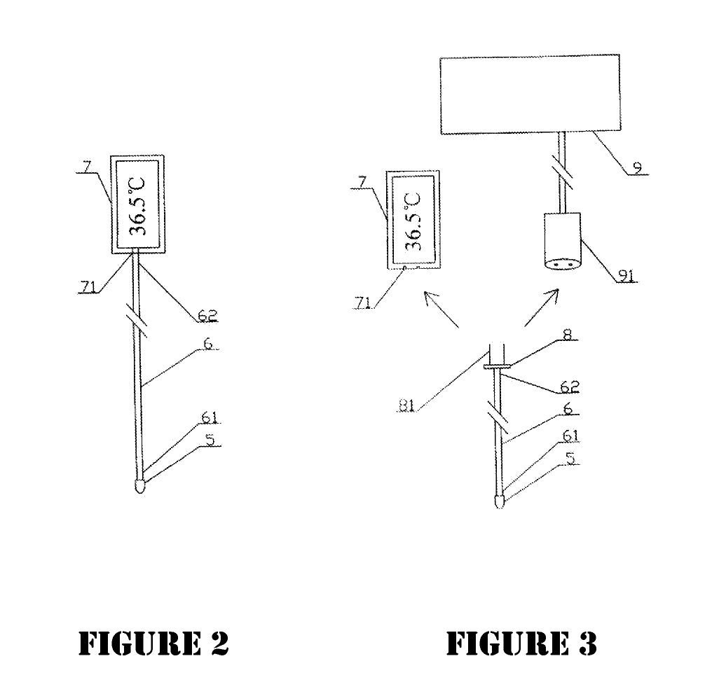

Said signal wire 6 has a probing end 61, which is connected to the temperature probe 5. Said signal wire has an external end 62, as shown in FIG. 2 and FIG. 3, that can be attached to a connecting socket 8, whose connector 81 then in turns connects to the temperature display unit 7, or to an external multi-function monitoring device 9 via an additional cord, having an interface 91.

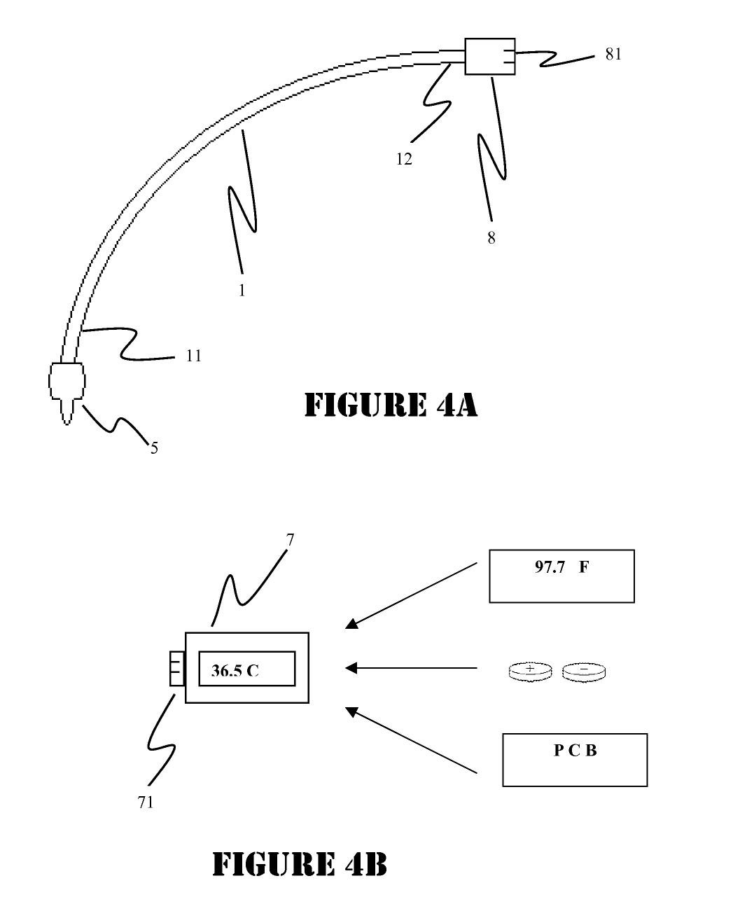

The in-body portion of present invention consists of the portion of the air tube 1 from the inside end 11 to the outside end 62, which ends with a connecting socket 8, containing a connector 81. See FIG. 4a and FIG. 4b for such suggested 2-unit construction.

The outside-body portion of present invention has a connector port 71 for receiving the connector 81, to transmit temperature readout signals to display unit 7, as shown in FIG. 4b.

A battery compartment can be built near the outside end 12 of said air tube 1, as well as a switch in association with a printed circuit board, so that the device can be turned on/off by the switch. This can be done on the outside-body portion, as suggested in FIG. 4b.

As such, this type of integrated temperature probe with display unit can be applied to other implementations of the same nature which should be considered within the scope of present invention.

What is claimed is:

1. An LMA device with integrated core temperature monitoring and display, comprising:
An in-body portion having an air tube containing an inside end and an outside end;
An air sac located near the inside end of said air tube;
A temperature probe located near the perimeter edges and along the dorsal side of said air sac;
A signal wire inside said air tube, connecting said temperature probe to a connecting socket near said outside end;
An outside body portion having a temperature display unit having a connector for attaching to the connecting socket on said air tube; and,
An air pump connected to said air sac, whereby said air pump can be operated to inflate said air sac by a pumping tube connected to said air sac.

2. The device of claim 1, wherein said signal wire further having a probing end connected to said temperature display unit and an external end that contains a wire connecting socket with a connector for connecting to either the connector port of said temperature display unit or an interface for an external display.

3. The device of claim 1, wherein said temperature probe is embedded on the sac wall at about one-third of the way from the very distal tip of the air sac.

4. The device of claim 1, wherein two or more temperature probes may be placed evenly around the edge of the sac wall for achieving averaged temperature reading.

5. The device of claim 1, wherein said temperature display unit further having a digital display readout, battery compartment and a switch in association with a printed circuit board.

6. An LMA device with integrated core temperature probe, comprising:
An in-body portion having an air tube containing an inside end and an outside end;
An air sac located near the inside end of said air tube;
A temperature probe located near the perimeter edges and along the dorsal side of said air sac;
A signal wire inside said air tube, connecting said temperature probe to a connecting socket near said outside end;
An outside body portion having a connector for attaching to the connecting socket on said air tube;
An air pump connected to said air sac, whereby said air pump can be operated to inflate said air sac by a pumping tube connected to said air sac; and,
A signal wire inside said air tube, connecting said temperature probe to a connecting socket near said outside end, whereby said connecting socket allows the temperature sensing and reading to be sent to an external monitor by an external cord.

7. The device of claim 6, wherein said temperature display unit further having two or more temperature probes placed evenly around the edge of the sac wall for achieving averaged temperature reading.
